(19) United States
(12) Patent Application Publication
(10) Pub. No.: US 2004/0030352 A1
(43) Pub. Date: Feb. 12, 2004

(54) Device for staunching vagina bleeding

(76) Inventors: Timothy Mary McGloughlin, Limerick (IE); Paul Anthony Slattery, Killaloe (IE); John Drumm, Limerick (IE); Gerard Burke, Limerick (IE); Delan Patrick Lyons, Limerick (IE); Mark Charles Skehan, Limerick (IE); Hugh Delan Flood, Clonlara (IE); John Joseph Quigley, Limerick (IE)

Correspondence Address: SUGHRUE MION, PLLC, 2100 PENNSYLVANIA AVENUE, N.W., WASHINGTON, DC, 20037, US

(21) Appl. No.: 10/380,947

(22) PCT Filed: Sep. 20, 2001
(86) PCT No.: PCT/IE01/00122

(30) Foreign Application Priority Data
Sep. 20, 2000 (IL) S2000/0761

(51) Int. Cl. A61M 29/00

(52) U.S. Cl. 606/193; 604/102.01

(57) ABSTRACT

A device (1) for staunching bleeding from an incision (2) made in the wall (3) of the vagina (4) of a subject comprises an inflatable member (8) carried on a tubular carrier member (5) which is inflatable within the vagina (4) for applying pressure to the wall (3) of the vagina (4) for urging a portion of the wall (3) adjacent the incision (2) against an adjacent portion of the anatomy, for example, the pubic bone (41) for staunching bleeding from the incision (2). The inflatable member (8) is inflated through the carrier member (5) through a releasable one-way valve (27) which retains inflatable medium, for example, air in the inflatable member (8) and which also facilitates exhausting of the inflatable medium from the inflatable member (8) for removal of the inflatable member (8) from the vagina (4). A drain tube (30) extending through the carrier member (5) drains blood and other fluids from the vagina (4) between the inflatable member (8) and the cervix (33). Monitoring the rate of blood being drained through the drain tube (30) indicates the rate at which bleeding from the incision (2) is being staunched.

The present invention relates to a device for staunching bleeding in a subject following vaginal surgery. The invention also relates to use of the device for staunching bleeding in a subject following vaginal surgery, and the invention further relates to a method for staunching bleeding in a subject following vaginal surgery.

Surgical procedures requiring an incision to be made in the vagina wall are commonplace. These procedures may be carried out on the vagina itself, or may involve incising the vagina wall to gain access to another organ or part of the body, for example, the urethra. This latter type of surgery is known as transvaginal surgery. On completion of the surgery the incision in the vagina wall is stitched. However, in many cases the stitching may be insufficient to staunch bleeding, and excessive bleeding may occur following such surgical procedures.

There is therefore a need for a device for staunching bleeding in a subject following vaginal surgery. There is also a need for use of such a device for staunching bleeding in a subject following vaginal surgery, and there is a need for a method for staunching bleeding in a subject following vaginal surgery.

The present invention is directed towards providing such a device, use of the device and a method for staunching bleeding in a subject following vaginal surgery.

According to the invention there is provided a device for staunching bleeding from an incision in the wall of the vagina of a subject, wherein the device comprises an expandable member for inserting into the vagina, the expandable member being operable in a non-expanded state for facilitating insertion into the vagina, and in an expanded state within the vagina for staunching the bleeding, a carrier means carrying the expandable member for inserting and for guiding the expandable member into the vagina, and a communicating means for communicating with the expandable member through the vagina for expanding the expandable member in the vagina.

In one embodiment of the invention the expandable member is adapted for defining the shape of the vagina when in the expanded state within the vagina.

Preferably, the expandable member when in the expanded state is an elongated member having an outer surface extending between a proximal end for locating towards the vagina entrance and a distal end for locating towards the cervix, the outer surface being adapted for engaging the wall of the vagina, and the expandable member defines a longitudinally extending central axis when in the expanded state.

In one embodiment of the invention the expandable member when in the expanded state diverges outwardly from the proximal end to the distal end. Preferably, the expandable member when in the expanded state defines a cone angle in the range of 5° to 45°. Advantageously, the expandable member when in the expanded state defines a cone angle in the range of 5° to 20°. Ideally, the expandable member when in the expanded state defines a cone angle of approximately 10°.

In one embodiment of the invention at least one rib extends circumferentially around and outwardly of the expandable member when the expandable member is in the expanded state for retaining the expandable member in the vagina. Preferably, a plurality of axially spaced apart circumferentially extending ribs extend around the expandable member. Advantageously, each rib is of arcuate cross-section. Ideally, each rib is formed by the material of the expandable member.

In one embodiment of the invention the outer diameter of the ribs progressively increases from the proximal end to the distal end of the expandable member.

Alternatively, the expandable member is of ovoid shape when in the expanded state.

Preferably, the expandable member is of circular transverse cross-section when in the expanded state.

In one embodiment of the invention the expandable member is an inflatable member. Preferably, the expandable member is of a resilient material so that the expandable member is expandable to selectable sizes when in the expanded state, and advantageously, the expandable member is of a flexible material.

In one embodiment of the invention the carrier means comprises an elongated semi-rigid carrier member, and preferably, the carrier member is of limited flexibility.

In one embodiment of the invention the carrier member forms the communicating means. Preferably, the carrier member comprises an elongated tubular member having an elongated bore communicating with the expandable member for inflating thereof.

In one embodiment of the invention the carrier member terminates in an inflating medium inlet for accommodating inflating medium into the bore of the carrier member for delivery of the inflating medium to the expandable member.

In another embodiment of the invention a valving means is provided in the carrier member for retaining inflating medium in the expandable member. Preferably, the valving means is a one-way valve adapted for accommodating inflating medium into the expandable member, and for preventing reverse flow of inflating medium for retaining the expandable member in the expanded state. Advantageously, the valving means comprises a release means for selectively releasing the valving means for permitting reverse flow of inflating medium for deflating the expandable member.

In another embodiment of the invention a draining means is provided for draining fluid from the vagina. Preferably, the draining means terminates in a drain inlet for locating within the vagina for receiving fluid to be drained from the vagina. Preferably, the drain inlet is located adjacent the distal end of the expandable member. Advantageously, the draining means extends through the expandable member.

Alternatively, the drain inlet is located adjacent the proximal end of the expandable member.

In one embodiment of the invention the draining means extends through the communicating means. Preferably, the draining means extends from the communicating means intermediate the expandable member and the inflating medium inlet. Advantageously, the draining means extends from the communicating means adjacent the valving means.

In one embodiment of the invention the draining means comprises an elongated tubular member.

In another embodiment of the invention the draining means terminates in a drain outlet remote from the drain inlet.

In a further embodiment of the invention the expandable member is adapted for urging a portion of the wall of the vagina adjacent the incision against an adjacent portion of the anatomy of the subject for staunching bleeding from the incision.

Additionally, the invention provides use of the device according to the invention for staunching bleeding from an incision made in the wall of the vagina of a subject, the expandable member being inserted into the vagina in the non-expanded state and being expanded therein to the expanded state for urging the portion of the wall of the vagina adjacent the incision against an adjacent portion of the anatomy of the subject.

Further the invention provides use of a device for staunching bleeding from an incision in the wall of the vagina of a subject, the device comprising an expandable member for inserting into the vagina, the expandable member being operable in a non-expanded state for facilitating insertion into the vagina, and in an expanded state within the vagina for staunching the bleeding, a carrier means carrying the expandable member for inserting and for guiding the expandable member into the vagina, and a communicating means for communicating with the expandable member through the vagina for expanding the expandable member in the vagina, the expandable member being inserted into the vagina in the non-expanded state, and being expanded in the vagina to the expanded state for urging a portion of the wall of the vagina adjacent the incision against an adjacent part of the anatomy of the subject for staunching bleeding from the incision.

In one embodiment of the invention the expandable member is expanded in the vagina for urging the portion of the wall of the vagina adjacent the incision against the pubic bone of the subject for staunching bleeding from the incision.

Further the invention provides a method for staunching bleeding from an incision in the wall of the vagina of a subject, the method comprising the steps of inserting an expandable member into the vagina and expanding the expandable member to bear on the wall of the vagina adjacent the incision for staunching the bleeding therefrom.

In one embodiment of the invention a portion of the wall of the vagina adjacent the incision is urged against an adjacent part of the anatomy of the subject by the expandable member for staunching the bleeding.

In another embodiment of the invention the portion of the wall of the vagina adjacent the incision is urged against the pubic bone of the subject by the expandable member.

In a still further embodiment of the invention the method further comprises the step of draining fluid from the vagina through a draining means extending into the vagina.

In another embodiment of the invention the draining means is inserted into the vagina to a location intermediate the expandable member and the cervix.

The advantages of the invention are many. The device according to the invention readily staunches bleeding from an incision in the vagina wall. This is achieved by virtue of the fact that the expandable member urges the portion of the vagina wall adjacent the incision against an adjacent portion of the anatomy of the subject, thereby squeezing the portion of the vagina wall adjacent the incision, and in turn closing blood vessels adjacent the incision. By virtue of the fact that the device comprises an expandable member, insertion of the device into the vagina is readily facilitated when the expandable member is in the non-expanded state. A further advantage of providing the device with an expandable member is that manoeuvring of the expandable member into the optimum position within the vagina prior to expanding thereof is facilitated. A particularly important advantage of the invention is achieved when the expandable member is an inflatable member in that manoeuvrability of the expandable member into the optimum position is further enhanced. By partially inflating the expandable member after initial insertion into the vagina, the partially inflated expandable member can more easily be manoeuvred to the optimum location within the vagina, at which stage final inflation of the expandable member is carried out.

By providing the expandable member of a resilient material, the expandable member can be inflated to different sizes for varying the pressure on the wall of the vagina. By providing a releasable valving means which permits reverse flow of the inflating medium from the expandable member, a further advantage is achieved in that as the bleeding is being progressively staunched, inflating medium can be periodically released from the expandable member for reducing the size of the expandable member, and thus decrementally reducing the pressure being applied to the wall of the vagina.

A further advantage of the invention is achieved by providing the expandable member of shape whereby the expandable member diverges from the proximal end to the distal end, in that retention of the expandable member within the vagina is enhanced. Providing at least one circumferentially extending rib extending radially outwardly of the expandable member further facilitates retention of the expandable member within the vagina.

The provision of the drain means is particularly advantageous, in that it facilitates draining of fluid, in particular, blood from the vagina prior to complete staunching of the bleeding. A particularly advantageous arrangement of the draining means is provided when the drain inlet of the draining means is located towards the distal end of the expandable member. This arrangement allows blood and other fluids which collect upstream of the expandable member in the vagina, in other words, between the expandable member and the cervix, to be readily drained from the vagina. This arrangement of the draining means also facilitates monitoring of the progress of staunching the bleeding from the incision by monitoring the rate at which blood is draining through the draining means.

The invention will be more clearly understood from the following description of some preferred embodiments thereof, which are given by way of example only, with reference to the accompanying drawings, in which:

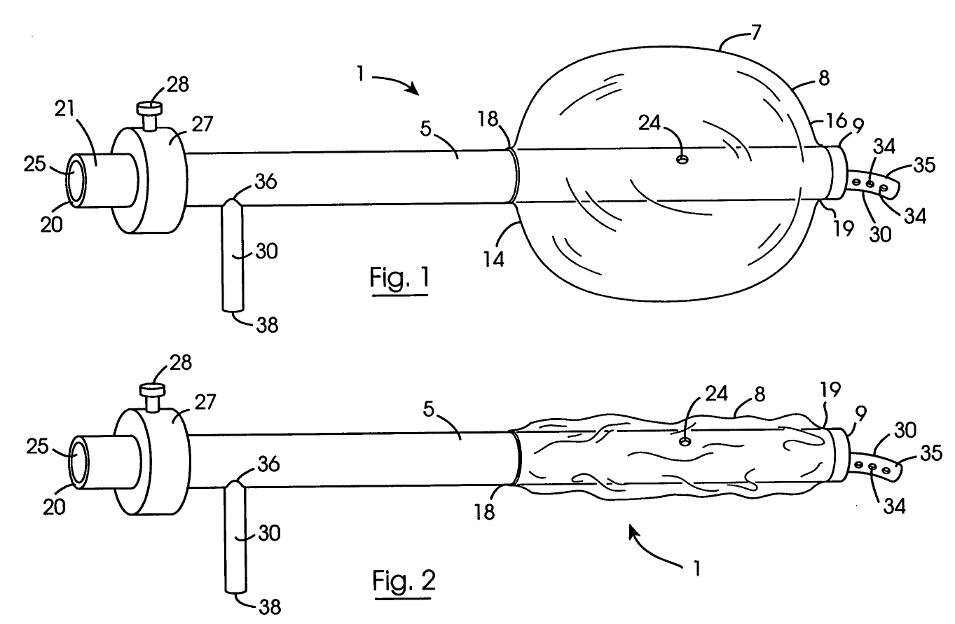

FIG. 1 is a perspective view of a device according to the invention for staunching bleeding from an incision in the vagina wall of a subject,

FIG. 2 is a view similar to FIG. 1 of the device of FIG. 1 illustrated in a different state,

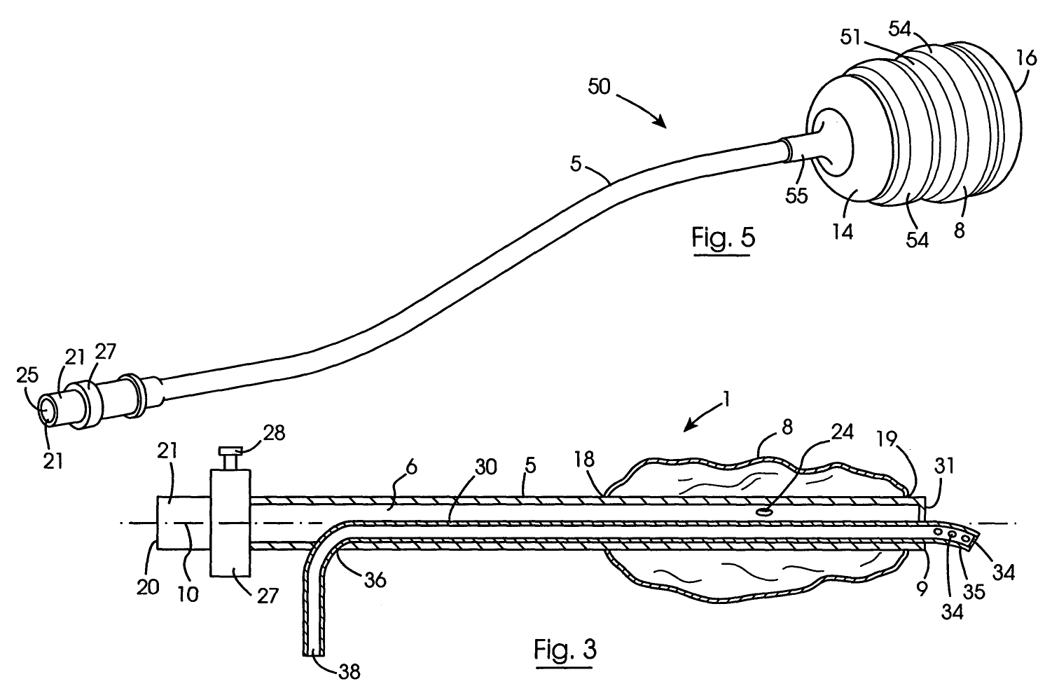

FIG. 3 is a transverse cross-sectional side elevational view of the device of FIG. 1,

FIG. 4 is a transverse cross-sectional side elevational view of the device of FIG. 1 in use,

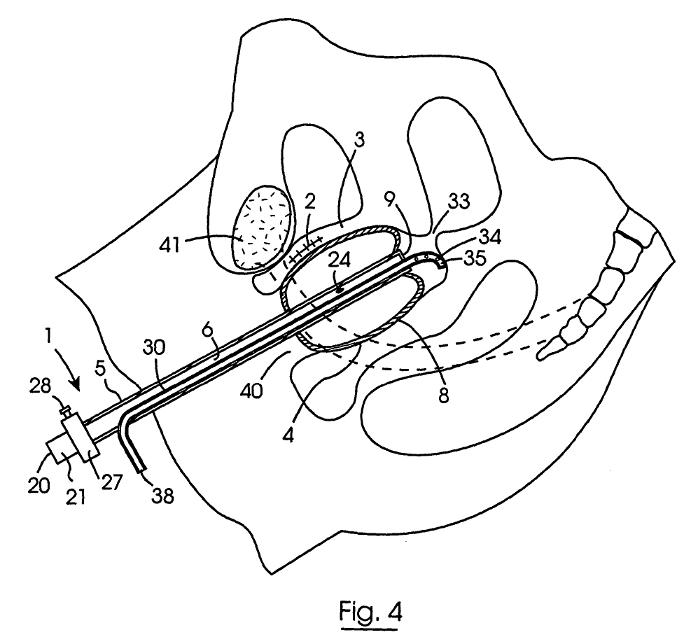

FIG. 5 is a perspective view of a device according to another embodiment of the invention for staunching bleeding from an incision in the vagina wall of a subject,

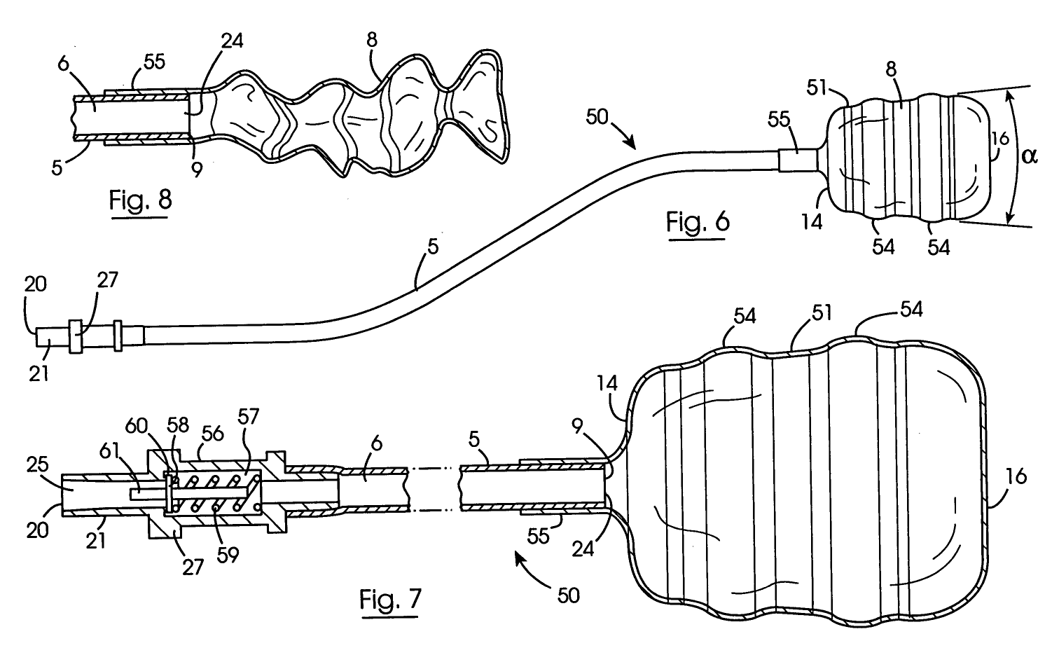

FIG. 6 is a side elevational view of the device of FIG. 5,

FIG. 7 is a transverse cross-sectional side elevational view of the device of FIG. 5, and

FIG. 8 is a transverse cross-sectional side elevational view of a detail of the device of FIG. 5 illustrated in a different state.

Referring to the drawings and initially to FIGS. 1 to 4, there is illustrated a device according to the invention indicated generally by the reference numeral 1 for staunching bleeding from an incision 2 made in a wall 3 of a vagina 4 of a subject following vaginal surgery. The incision 2 may result from a surgical procedure is carried out on the vagina itself, or may be an incision formed in the wall 3 of the vagina 4 to provide access to another part or organ of the body of the subject. The device 1 comprises a carrier means, namely, an elongated tubular carrier member 5 having a bore 6 extending therethrough, and an expandable member, namely, an inflatable member 8 carried on and located around the carrier member 5 towards an end 9 thereof. The inflatable member 8 is operable between a non-expanded state, namely, a deflated state collapsed around the carrier member 5, see FIGS. 2 and 3, for facilitating insertion into the vagina 4, and an expanded state, namely, an inflated state, see FIGS. 1 and 4, for applying pressure to the wall 3 of the vagina 4 adjacent the incision 2 when inflated within the vagina 4 for staunching bleeding from the incision 2. The carrier member 5 is of a semi-rigid resilient plastics material with limited flexibility for guiding the inflatable member 8 into and through the vagina 4 to the incision 2. The inflatable member 8 defines an outer surface 7 for engaging the wall 3 of the vagina 4 when inflated, is of resilient flexible plastics film material for facilitating inflation of the inflatable member 8 to different inflated sizes for facilitating varying pressure on the wall 3 of the vagina 4 as the bleeding from the incision 2 is being progressively staunched.

In this embodiment of the invention the inflatable member 8 when inflated defines a longitudinally extending central axis 10, and is sealably secured to the carrier member 5 at a proximal end 14 and at a distal end 16 at axially spaced apart locations 18 and 19, respectively, on the carrier member 5. The carrier member 5 as well as carrying the inflatable member 8 also acts as a communicating means for facilitating delivery of an inflating medium, typically, air or a saline solution through the bore 6 into the inflatable member 8 for inflating thereof. An inflating medium inlet 20 at an end 21 of the carrier member 5 remote from the end 9 accommodates the inflating medium into the bore 6 of the carrier member 5. An inflating medium outlet 24 through the wall of the carrier member 5 intermediate the locations 18 and 19 thereof accommodates inflating medium from the bore 6 of the carrier member 5 into the inflatable member 8. The inflating inlet 20 terminates in a port 25 which is internally tapered and shaped for engaging a lure or record tapered portion of a syringe of the type typically used in conjunction with a hypodermic needle for facilitating pumping of inflating medium through the bore 6 of the carrier member 5 into the inflatable member 8.

A valving means, namely, a releasable one-way valve 27 is located in the carrier member 5 adjacent the inlet 20 for permitting the flow of inflating medium into the inflatable member 8, and also for retaining the inflating medium in the inflatable member 8 for retaining the inflatable member 8 inflated within the vagina 4. However, the one-way valve 27 is releasable for facilitating exhausting of the inflating medium from the inflatable member 8 for facilitating removal of the inflatable member 8 from the vagina when bleeding has been completely staunched. A trigger button 28 on the one-way valve 27 is provided for releasing the one-way valve 27 for exhausting the inflating medium from the inflatable member 8. Additionally, the trigger button 28 may be momentarily operated for partially exhausting the inflating medium from the inflatable member 8, for facilitating a decremental reduction of the pressure being applied to the wall 3 of the vagina 4 by the inflatable member 8 as bleeding from the incision is being progressively staunched. Periodic momentary operation of the trigger button 28 facilitates progressive decremental reduction of the pressure applied to the wall 3 of the vagina 4 as bleeding is being progressively staunched.

A draining means, namely, a drain tube 30 extends through the bore 6 of the carrier member 5 and sealably exits through an end cap 31 in the end 9 of the carrier member 5 at the distal end 16 of the inflatable member 8 for draining fluid, for example, blood, mucus and other fluids from the incision 2 and elsewhere. Such blood and fluids would otherwise collect in the vagina 4 between the inflatable member 8 and the cervix 33. A plurality of drain inlet ports 34 in a portion 35 of the drain tube 30 extending from the end cap 31 receive the blood and other fluids from the vagina. The drain tube 30 exits through an opening 36 in the carrier member 5 intermediate the valve 27 and the inflatable member 8, and terminates in a drain outlet port 38 for delivering blood and other fluids drained from the vagina 4 into a suitable collecting vessel (not shown), for exaple, a bag or the like.

The carrier member 5 sealably engages the drain tube 30 as it passes through the opening 36 for providing an airtight seal. Similarly, the end cap 31 sealably engages the drain tube 30 for likewise forming an airtight seal. The proximal and distal ends 14 and 16 of the inflatable member 8 sealably engage the carrier member 5 for also forming an airtight seal.

In use, after the incision 2 in the wall 3 of the vagina 4 has been stitched, the inflatable member 8 in the deflated state is inserted into the vagina 4 by urging the carrier member 4 and the inflatable member 8 through the vagina opening 40. When the inflatable member 8 is wholly within the vagina 4, and is approximately aligned with the surgical site adjacent the stitched incision 2, the port 25 of the inflating medium inlet 20 is connected to a suitably sized syringe and the inflating medium, typically air or a saline solution is pumped into the inflatable member 8 through the carrier member 5 until the inflatable member 8 is reasonably well inflated. If the inflatable member is not accurately aligned with the stitched incision 2, further alignment is then carried out, and the inflatable member 8 is then inflated to the desired size for applying the appropriate amount of pressure to the wall 3 of the vagina 4. If the incision 2 had been made to provide access to the urethra, the inflatable member 8 is inflated so that the portion of the wall 3 of the vagina adjacent the stitched incision 2 is urged against the pubic bone 41 of the subject with a sufficient pressure for commencing staunching of bleeding from the incision 2. Blood and other fluids in the vagina between the inflatable member 8 and the cervix 33 are drained from the vagina 4 through the drain tube 30, and collected in a suitable container, for example, a bag or the like.

As the bleeding from the incision 2 is progressively staunched, if desired the inflatable member 8 may be progressively deflated by decrementally exhausting inflating medium from the inflatable member 8 through the one-way valve 27. When bleeding has been completely staunched, the inflatable member 8 is completely deflated through the one-way valve 27 and is removed from the vagina by urging the carrier member 5 downwardly through the vagina opening 40. The rate at which the bleeding from the stitched incision is being staunched can be monitored by monitoring the rate at which blood is being drained from the vagina 4 through the drain tube 30.

Referring now to FIGS. 5 to 8, there is illustrated a device according to another embodiment of the invention indicated generally by the reference numeral 50 for staunching bleeding from an incision 2 in a wall 3 of a vagina of a subject following vaginal surgery. The device 50 is substantially similar to the device 1 and similar components are identified by the same reference numerals. The main difference between the device 50 and the device 1 is that the device 50 is provided without a drain tube, and additionally the carrier member 5 does not extend through the inflatable member 8. The one-way valve 27 is of a different construction to that of the device 1, and in particular, the shape of the inflatable member 8 of the device 50 is different to that of the inflatable member 8 of the device 1.

In this embodiment of the invention the inflatable member 8 when inflated defines an outer surface 51 of circular transverse cross-section which diverges from its proximal end 14 to its distal end 16 and defines a cone angle α of approximately 10° for facilitating retention of the inflatable member 8 in the vagina. Additionally, retention of the inflatable member 8 in the vagina 4 is also enhanced by the provision of two circumferentially extending ribs 54 which extend circumferentially around the inflatable member 8 and extend radially outwardly therefrom. The circumferential ribs 54 are of arcuate cross-section when viewed in side elevation, and are formed in the film material of the inflatable member 8 during formation of the inflatable member 8, which typically is formed by blow moulding.

The carrier member 5 extends into the inflatable member 8 and terminates at its proximal end 14. In this embodiment of the invention the inflating medium outlet 24 from the bore 6 of the carrier member 5 into the inflatable member 8 is defined by the bore 6 at the end 9 of the carrier member 5. A flange 55 extending from the proximal end 14 of the inflatable member 8 sealably and securely engages the carrier member 5 for connecting the inflatable member 8 to the carrier member 5.

Referring now in particular to FIG. 7, the one-way valve 27 of the device 50 will now be described. In this embodiment of the invention the valve 27 also forms the inflating medium inlet 20 and the inlet port 25. The one-way valve 27 comprises a valve housing 56 which defines a valving chamber 57 within which a valving member 58 is spring urged by a compression spring 59 against a valve seat 60 formed in the valve housing 56. A spindle 61 extending from the valving member 58 into the port 25 is provided for urging the valving member 58 off the seat 60 for releasing the one-way valve 57 for exhausting inflating medium from the inflatable member 8.

Otherwise, the device 50 is similar to the device 1 and its use and operation is likewise similar.

While the device 50 has been described without a drain tube, it will be readily apparent to those skilled in the art that the device 50 could be provided with a drain tube, and in which case, in general, it is envisaged that the drain tube 30 would extend substantially axially through the inflatable member 8 and extend sealably therefrom at the distal end 16 of the inflatable member 8.

It will also be appreciated that the device 1 could be provided with a one-way valve similar to that provided with the device 50. Needless to say, it will be appreciated that the carrier member could be provided to extend substantially axially through the inflatable member of the device 50.

While the devices according to the invention have been described for use with staunching bleeding from a stitched incision in the wall of the vagina which had been made to provide access to the urethra, it will be readily apparent to those skilled in the art that the device may be used for staunching bleeding from any incision formed in any other location in the wall of the vagina. Such incision may be formed in the wall of the vagina for providing access to other parts or organs of the body of the subject, or indeed, for carrying out a procedure on the vagina itself. In which case, the inflatable member would be inflated to the appropriate size for providing an appropriate pressure to the wall of the vagina for urging the portion of the vagina wall adjacent the stitched incision into engagement with an adjacent part of the anatomy of the subject for staunching the bleeding.

While the devices 1 have been described as being inflated by an inflating medium provided by air or a saline solution, any other suitable inflating medium may be used, for example, water, or the like. Additionally, the source of the inflating medium may be any other suitable source besides a syringe. It is also envisaged that irrigation of the vagina and indeed the incision may be carried out through the drain tube 30. Such irrigation may be carried out using water, a saline solution or indeed, any other suitable irrigating medium.

It is envisaged that while in the two embodiments of the invention the expandable member has been described as being an inflatable member, any other suitable expandable member which is expandable from a non-expanded state to an expanded state could be used.

It is also envisaged that the outer surface of the inflatable member may be coated with any suitable preparation for assisting in staunching the bleeding, and also for protecting the incision against bacteria, for example, the inflating medium could be coated with a coagulating preparation or an antibacterial preparation so that when the inflating medium is inflated within the vagina, the preparation coated onto the inflatable medium would be transferred to the stitched incision.

1. A device for staunching bleeding from an incision (2) in the wall (3) of the vagina (4) of a subject, characterised in that the device (1) comprises an expandable member (8) for inserting into the vagina (4), the expandable member (8) being operable in a non-expanded state for facilitating insertion into the vagina (4), and in an expanded state within the vagina (4) for staunching the bleeding, a carrier means (5) carrying the expandable member (8) for inserting and for guiding the expandable member (8) into the vagina (4), and a communicating means (6) for communicating with the expandable member (8) through the vagina (4) for expanding the expandable member (8) in the vagina (4).

2. A device as claimed in claim 1 characterised in that the expandable member (8) is adapted for defining the shape of the vagina (4) when in the expanded state within the vagina.

3. A device as claimed in claim 1 or 2 characterised in that the expandable member (8) when in the expanded state is an elongated member having an outer surface (7, 51) extending between a proximal end (14) for locating towards the vagina entrance (40) and a distal end (16) for locating towards the cervix (33), the outer surface (7, 51) being adapted for engaging the wall (3) of the vagina (4).

4. A device as claimed in claim 3 characterised in that the expandable member (8) defines a longitudinally extending central axis when in the expanded state.

5. A device as claimed in claim 3 or 4 characterised in that the expandable member (8) when in the expanded state diverges outwardly from the proximal end (14) to the distal end (16).

6. A device as claimed in claim 5 characterised in that the expandable member (8) when in the expanded state defines a cone angle (α) in the range of 5° to 45°.

7. A device as claimed in claim 5 or 6 characterised in that the expandable member (8) when in the expanded state defines a cone angle (α) in the range of 5° to 20°.

8. A device as claimed in any of claims 5 to 7 characterised in that the expandable member (8) when in the expanded state defines a cone angle (α) of approximately 10°.

9. A device as claimed in any preceding claim characterised in that at least one rib (54) extends circumferentially around and outwardly of the expandable member (8) when the expandable member (8) is in the expanded state for retaining the expandable member (8) in the vagina (4).

10. A device as claimed in claim 9 characterised in that a plurality of axially spaced apart circumferentially extending ribs (54) extend around the expandable member (8).

11. A device as claimed in claim 9 or 10 characterised in that each rib (54) is of arcuate cross-section.

12. A device as claimed in any of claims 9 to 11 characterised in that each rib (54) is formed by the material of the expandable member (8).

13. A device as claimed in any of claims 9 to 12 characterised in that the outer diameter of the ribs (54) progressively increases from the proximal end (14) to the distal end (16) of the expandable member (8).

14. A device as claimed in any of claims 1 to 4 characterised in that the expandable member (8) is of ovoid shape when in the expanded state.

15. A device as claimed in any preceding claim characterised in that the expandable member (8) is of circular transverse cross-section when in the expanded state.

16. A device as claimed in any preceding claim characterised in that the expandable member (8) is an inflatable member.

17. A device as claimed in any preceding claim characterised in that the expandable member (8) is of a resilient material so that the expandable member (8) is expandable to selectable sizes when in the expanded state.

18. A device as claimed in any preceding claim characterised in that the expandable member (8) is of a flexible material.

19. A device as claimed in any preceding claim characterised in that the carrier means (5) comprises an elongated semi-rigid carrier member (5).

20. A device as claimed in any preceding claim characterised in that the carrier member (8) is of limited flexibility.

21. A device as claimed in any preceding claim characterised in that the carrier member (8) forms the communicating means (6).

22. A device as claimed in claim 21 characterised in that the carrier member (5) comprises an elongated tubular member (5) having an elongated bore (6) communicating with the expandable member (8) for inflating thereof.

23. A device as claimed in claim 22 characterised in that the carrier member (5) terminates in an inflating medium inlet (20) for accommodating inflating medium into the bore (6) of the carrier member (5) for delivery of the inflating medium to the expandable member (8).

24. A device as claimed in claims 22 or 23 characterised in that a valving means (27) is provided in the carrier member (5) for retaining inflating medium in the expandable member (8).

25. A device as claimed in claim 24 characterised in that the valving means (27) is a one-way valve adapted for accommodating inflating medium into the expandable member (8), and for preventing reverse flow of inflating medium for retaining the expandable member (8) in the expanded state.

26. A device as claimed in claim 24 or 25 characterised in that the valving means (27) comprises a release means (61) for selectively releasing the valving means (27, 60) for permitting reverse flow of inflating medium for deflating the expandable member (8).

27. A device as claimed in any preceding claim characterised in that a draining means (30) is provided for draining fluid from the vagina (4).

28. A device as claimed in claim 27 characterised in that the draining means (30) terminates in a drain inlet (34) for locating within the vagina for receiving fluid to be drained from the vagina (4).

29. A device as claimed in claim 28 characterised in that the drain inlet (34) is located adjacent the distal end (16) of the expandable member (8).

30. A device as claimed in any of claims 27 to 29 characterised in that the draining means (30) extends through the expandable member (8).

31. A device as claimed in claim 28 characterised in that the drain inlet (34) is located adjacent the proximal end (14) of the expandable member (8).

32. A device as claimed in any of claims 27 to 31 characterised in that the draining means (30) extends through the communicating means (6).

33. A device as claimed in claim 32 characterised in that the draining means (30) extends from the communicating means (6) intermediate the expandable member (8) and the inflating medium inlet (20).

34. A device as claimed in claim 33 characterised in that the draining means (30) extends from the communicating means (6) adjacent the valving means (20).

35. A device as claimed in any of claims 27 to 34 characterised in that the draining means (30) comprises an elongated tubular member (30).

36. A device as claimed in any of claims 27 to 35 characterised in that the draining means (30) terminates in a drain outlet (38) remote from the drain inlet (34).

37. A device as claimed in any preceding claim characterised in that the expandable member (8) is adapted for urging a portion of the wall (3) of the vagina (4) adjacent the incision (2) against an adjacent portion (41) of the anatomy of the subject for staunching bleeding from the incision (2).

38. Use of a device as claimed in any preceding claim for staunching bleeding from an incision (2) made in the wall (3) of the vagina (4) of a subject, the expandable member (8) being inserted into the vagina (4) in the non-expanded state and being expanded therein to the expanded state for urging the portion of the wall (3) of the vagina (4) adjacent the incision (2) against an adjacent portion (41) of the anatomy of the subject.

39. Use of a device for staunching bleeding from an incision (2) in the wall (3) of the vagina (4) of a subject, the device (1) comprising an expandable member (8) for inserting into the vagina (4), the expandable member (8) being operable in a non-expanded state for facilitating insertion into the vagina (4), and in an expanded state within the vagina (4) for staunching the bleeding, a carrier means (5) carrying the expandable member (8) for inserting and for guiding the expandable member (8) into the vagina (4), and a communicating means (6) for communicating with the expandable member (8) through the vagina (4) for expanding the expandable member (8) in the vagina (4), the expandable member (8) being inserted into the vagina (4) in the non-expanded state, and being expanded in the vagina (4) to the expanded state for urging a portion of the wall (3) of the vagina (4) adjacent the incision (2) against an adjacent part (41) of the anatomy of the subject for staunching bleeding from the incision (2).

40. Use as claimed in claim 39 characterised in that the expandable member (8) is expanded in the vagina (4) for urging the portion of the wall (3) of the vagina (4) adjacent the incision (2) against the pubic bone (41) of the subject for staunching bleeding from the incision (2).

41. A method for staunching bleeding from an incision (2) in the wall (3) of the vagina (4) of a subject, the method comprising the steps of inserting an expandable member (8) into the vagina (4) and expanding the expandable member (8) to bear on the wall (3) of the vagina (4) adjacent the incision (2) for staunching the bleeding therefrom.

42. A method as claimed in claim 41 characterised in that a portion of the wall (3) of the vagina (4) adjacent the incision (2) is urged against an adjacent part (41) of the anatomy of the subject by the expandable member (8) for staunching the bleeding.

43. A method as claimed in claim 42 characterised in that the portion of the wall (3) of the vagina (4) adjacent the incision (2) is urged against the pubic bone (41) of the subject by the expandable member (8).

44. A method as claimed in any of claims 41 to 43 characterised in that the method further comprises the step of draining fluid from the vagina through a draining means (30) extending into the vagina (4).

45. A method as claimed in claim 44 characterised in that the draining means (30) is inserted into the vagina (4) to a location intermediate the expandable member (8) and the cervix (33).
